(19) United States
(12) Patent Application Publication
(10) Pub. No.: US 2008/0261297 A1
(43) Pub. Date: Oct. 23, 2008

(54) Assay Device

(75) Inventors: Jason Chaffey, South Australia (AU); Murray Stapleton, Victoria (AU)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: RMIT UNIVERSITY, VICTORIA (AU)

(21) Appl. No.: 11/914,543

(22) PCT Filed: May 17, 2006
(86) PCT No.: PCT/AU2006/000656

§ 371 Date: Mar. 4, 2008

(30) Foreign Application Priority Data
May 20, 2005 (AU) 2005902630

(51) Int. Cl. C12M 1/40; B01J 19/00; C12M 1/34

(52) U.S. Cl. 435/287.9; 422/68.1; 435/287.1; 73/61.41

(57) ABSTRACT

An assay device (1) having a rotatable platform (2) with a test chamber (6) and a sensor (20) which undergoes displacement when subject to a particular substance such as a chemical, biological species or other organism. The sensor is a cantilever beam (21) with a porous section (23) to enhance sensitivity.

### FIELD OF THE INVENTION

The present invention relates to an assay device and a cantilevered detector.

### BACKGROUND OF THE INVENTION

It is known to use compact discs (CDs) for chemical testing. The CDs are provided with micro-fluidic structure which defines various fluid input ports in communication with associated channels and fluid mixing chambers. In order to conduct a test, fluid is deposited into the input ports and the CD is spun so that the fluid is forced by centrifugal pumping through the relevant channels to the mixing chambers. Significantly modified CD optics and addressing technology can be used to capture images of specific mixing chambers to determine the test results of any chemical reaction within the chambers.

It is also known that microcantilever beams have been considered as a means for detecting the results of chemical reactions, but the limited sensitivity of the beams studied has not resulted in any widespread application of the technology.

### SUMMARY OF THE INVENTION

In accordance with the invention, there is provided an assay device having a rotatable platform with a test chamber and a sensor which undergoes displacement when subject to a particular substance such as a chemical, biological species or other organism.

Preferably, the sensor is a cantilever beam.

Preferably, the device includes microfluidic paths in communication with associated channels and the test chamber or an associated plurality of test chambers.

Preferably, the or each test chamber includes one or more cantilever beams.

Preferably, the sensor includes a porous section. The porous section can give the sensor a sensitivity which enables the presence of the particular substance i.e., a selected chemical, species or organism, to be detected.

Preferably, the sensor is functionalised with receptors, antibodies, antigens or enzymes which will selectively attract and bond with the particular substance to be detected.

Preferably, the porous section is coated with a gold layer which attaches the receptors to the beam, functionalising the beam for bonding with preselected species or organism within the fluid in the test chamber.

Preferably, the sensor includes a surface to be monitored, which is subject to displacement upon movement of the sensor, the position of the monitored surface being monitored by an apparatus into which the assay device is loaded.

Preferably, the surface is a reflective surface.

Preferably, the apparatus is a CD drive connected to a computer allowing the position of the reflective surface to be determined and displayed by the computer.

Preferably, the assay device includes a micro-fluidic system for conveying a test fluid from an inlet port to the test chamber containing the cantilever beam and on to a waste chamber.

Preferably, the waste chamber is separated from the test chamber by a micro-mechanical valve which is actuated above a threshold angular velocity of the device.

Preferably, the device can accept the whole fluid to be tested and includes a filter for filtering material from the whole fluid after insertion into the inlet port to provide fluid in a form suitable for testing. More preferably, the filter is formed of a porous silicon.

Preferably, the system includes provision for secondary chamber connected to the test chamber to allow the cycling of fluid between the secondary and test chamber.

Preferably, the device is in the form of a compact disc (CD).

In another aspect, there is provided a test apparatus for receiving an assay device, as described above, including a drive unit for rotating the device and a read unit for monitoring the sensor.

Preferably, the apparatus is adapted to display information derived from the read unit.

More preferably, the apparatus is in the form of CD drive and the read unit forms part of an existing optical read/write head of the CD drive.

More preferably, the apparatus is connected directly to a computer, on which is installed a computer program which controls the operation of the CD drive to initiate the filtering process, the transfer of fluid between chambers and the optical reading system to measure the displacement of the sensor.

Preferably, the assay process is initiated by using the computer to input data defining the test to be performed and presenting the results with this same identification.

In another aspect, there is provided a chemical assay method including introducing fluid to a sensing chamber on a rotatable platform, wherein the sensing chamber includes a sensor arranged for displacement upon detection of a particular substance, such as a selected molecule, within the chamber, and monitoring the sensor to detect the displacement.

In yet another aspect, there is provided a cantilever sensor, as described above.

### BRIEF DESCRIPTION OF THE DRAWINGS

The invention is now described, by way of non-limiting example only, with reference to the accompanying drawings in which:

FIG. 1 is a diagrammatic representation of a plan view of an assay device;

FIG. 2 is a diagrammatic cross-sectional view of a test apparatus;

FIG. 3a is a diagrammatic side view of a microcantilever;

FIG. 3b is a diagrammatic side view of the microcantilever, illustrating deflection;

FIG. 4 is a graph illustrating a relationship between resonant frequency and porosity of a cantilever;

FIG. 5 is a diagrammatic perspective view of a cantilever sensor and a read/write head of a CD drive;

FIG. 6 is a graph illustrating a relationship between intensity and time, for the purpose of detecting displacement of the sensor;

FIG. 7 is a flow chart of a test procedure; and

FIG. 8 is a graph illustrating comparative deflection of a porous and non-porous cantilever.

### DETAILED DESCRIPTION

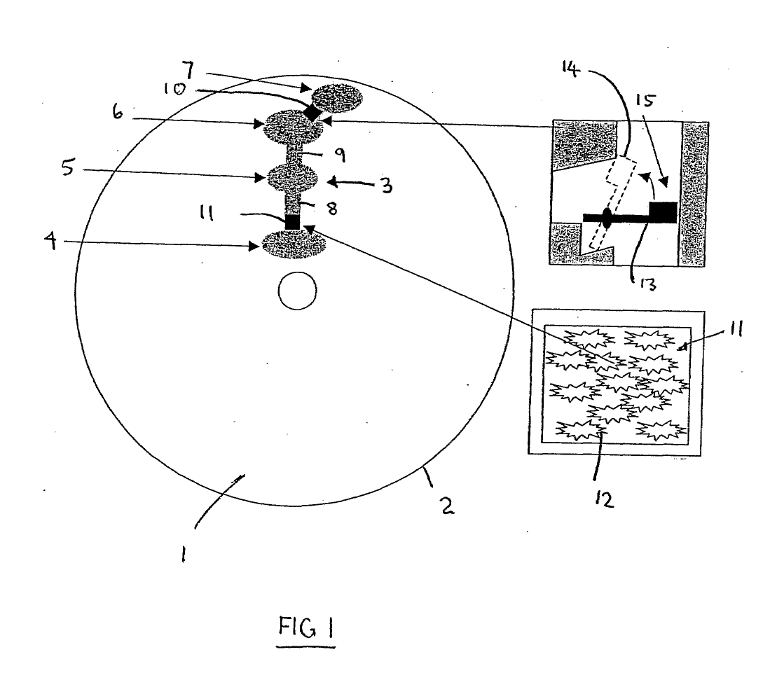

An assay device 1 is illustrated in FIG. 1 as including a rotatable platform 2, in the form of a compact disc (CD), with a microfluidic system 3 including an inlet port 4, a secondary chamber 5, a test chamber 6 and a waste chamber 7 interconnected by respective channels 8,9,10. A filter 11 is provided in one of the channels 8, adjacent the inlet port 4 for filtering material such as cellular material from the test fluid introduced into the inlet port 4. The filter 11 is preferably formed of porous silicon 12. A micro-mechanical valve 13 is also provided in the channel 10 separating the test chamber 6 and waste chamber 7. The valve 13 moves from a closed position, indicated by dashed lines 14, to an open position, indicated by arrow 15, when the angular velocity of the device 1 is above a predetermined threshold.

In operation, fluid is introduced into the inlet port 4 and the device 1 is rotated at a required speed to effect centrifugal pumping so that the fluid is forced through the channel 8 into the secondary chamber 5 and subsequently the test chamber 6 where a sensor is provided for the purpose of detecting the presence of a particular substance, such as a selected chemical, biological species or other organisms within the fluid. The device 1 is then rotated at higher angular velocity to open the valve 13 and allow the fluid to exit the test chamber 6.

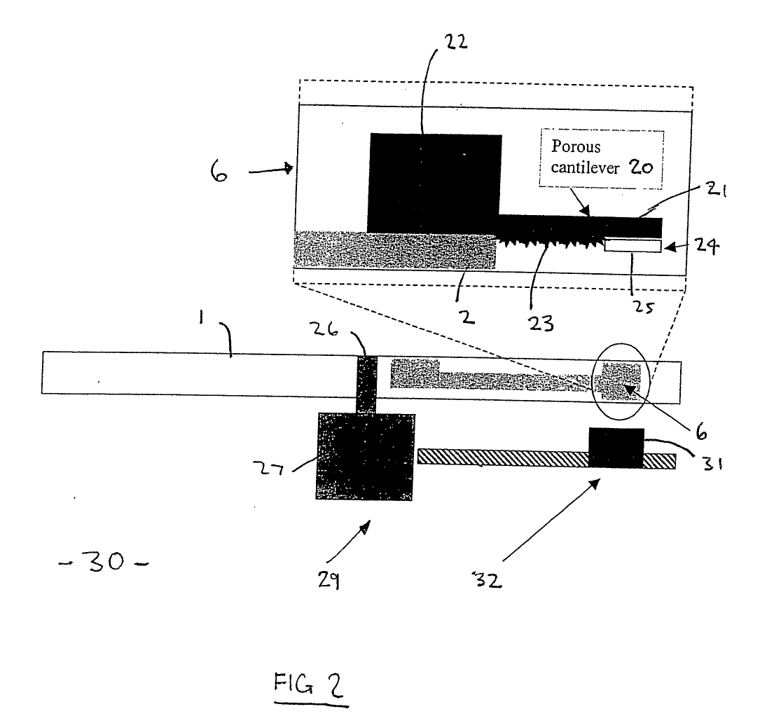

Referring now to FIG. 2, the test chamber 6 of the device 1 is shown in enlarged section as including a cantilever sensor 20, which projects from the platform 2 of the device 1. More specifically, the porous cantilever sensor 20 is formed of a beam 21 which projects from a silicon block 22 and includes a porous section 23 and a surface 24 formed of, for example, a section of gold 25 or other suitable metallic or reflective substance.

The device 1 is shown fitted on a spindle 26 of a drive unit 27 of a test apparatus 30, which is preferably in the form of a computer, with a CD drive 29 and the drive unit 27 forms part of the drive 29, together with a read unit 31, which monitors any displacement of the reference surface 24 and thereby the cantilever sensor 20. The read unit 31 preferably forms part of an existing read/write head 32 of the CD drive 31, without modification.

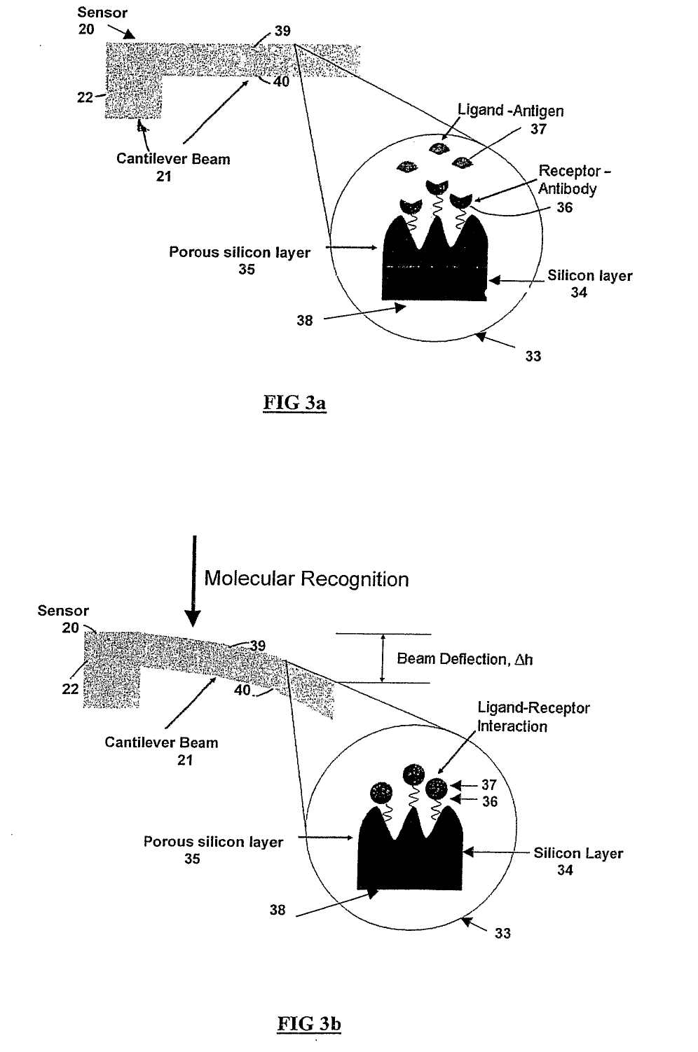

The structure of the cantilever sensor 20 is now described in more detail with reference to FIG. 3. FIG. 3a shows an enlarged part 33 of the sensor 20 as including a porous layer 35 and a silicon layer 34 both coated with gold which is provided with antibody receptors 36 for capturing molecules 37 such as antigen ligands. The binding of the molecules 37 to the receptors 36 will lead to a deflection of the beam 21, as illustrated in FIG. 3b, which can then be detected.

By forming the cantilever beam of porous material, the deflection is enhanced. More particularly, the characteristics of the cantilever sensor 20 rely on surface processes such as adsorption, desorption, surface reconstruction and reorganisation to induce a surface stress in the active surface layer of the cantilever beam 39. Modifying the surface stress on surface 39 of the beam 21 will induce a differential stress across the cantilever sensor 20, causing it to bend.

The curvature of the beam 21 is proportional to the differential stress gradient across the beam. Increasing surface stress on surface 39 compared to the surface 40 or layer 34 increases the differential stress gradient. Porous silicon at surface 39 can be used as the layer 35 to increase surface area and hence sensitivity. To the best of our knowledge, no research or development has been focussed on increasing the sensitivity of the cantilever based sensing technique by modifying the beam geometry or material structure. The beam 21 increases the maximum surface stress that can be induced by the chemical analyte by introducing the porous layer 35 and modifying the beam geometry.

Analysis and tests have shown that by modifying the beam, geometry and material structures as described, the increased beam deflection for increased porosity can be varied as shown in FIG. 8.

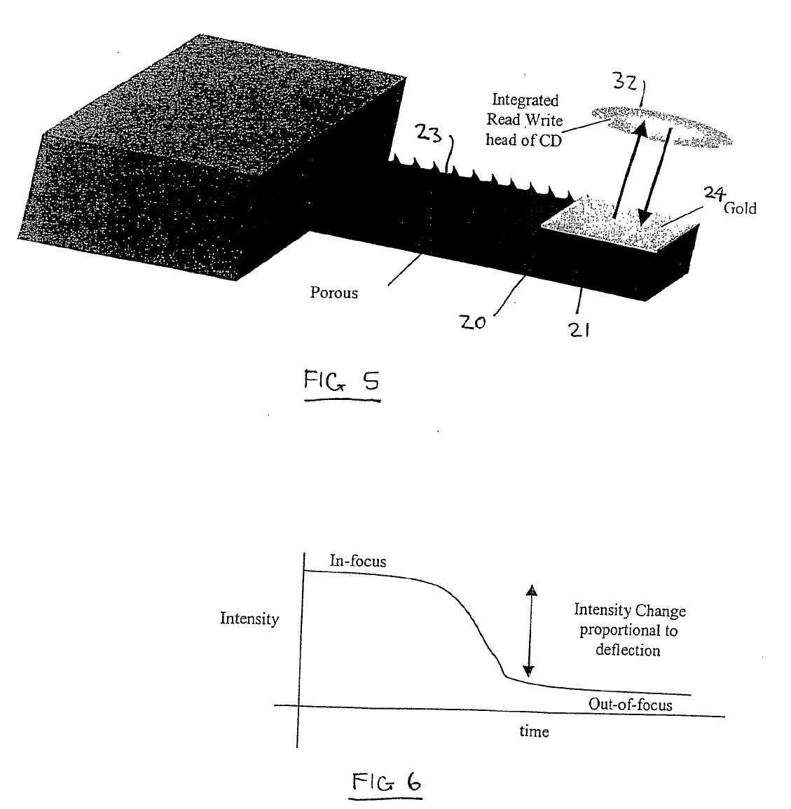

Accordingly, in FIG. 5, the sensor 20 allows for increased deflection of the cantilever beam 21, as compared to a conventional beam of the same thickness and length, by fabricating a porous section on surface 23 of the beam 21. This has three affects on the mechanical response of the beam 21:
1. It reduces the effective thickness of the beam where it is porous, reducing the second moment of inertia of the beam, making the beam less rigid;
2. The spring constant of the beam is also reduced where it is porous; and
3. The surface area of the beam is also increased due to the increased porosity of the cantilever beam of the cantilever beam.

These three physical affects have a combined effect to increase the deflection of the beam and sensitivity to surface-combination events over current cantilever based biosensors. Increasing the differential stress induced between the layers 35,34 of the beam, FIG. 3b leads to an increase in the deflection. Further to this, the surface area to be functionalised, i.e. provided with receptors for bonding with selected molecules, is increased, allowing for a greater density of functionalised groups to be attached to the surface, thereby increasing the sensitivity and induced surface stress for the same concentration of chemical or biological species.

This enables a more concentrated binding of the species and also enables less variation in deflection for the same chemical or species concentrate.

Another affect of modifying the geometry of the beam is that the resonant frequency of the beam is changed since the resonant frequency is a direct measure of the amount of porosity.

The resonant frequency change according to the beam geometry has the following relationship:

$$fo = \frac{1}{2 π \sqrt{\frac{k}{m}}}$$

where
fo=resonance frequency
k=spring constant
m=mass of the beam

A change of porosity changes the resonant frequency of the cantilever beam 21 and is an additional sensing capability of the sensor, which could be applied to detection of corrosion or chemical reaction caused by fluid, for example, measurement of corrosion on a marine vessel or detection of acid rain or similar events for environmental monitoring.

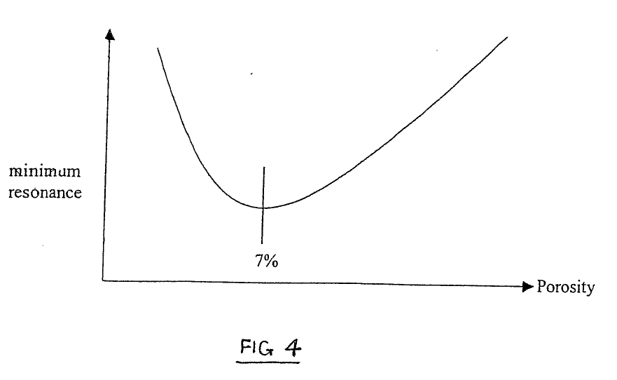

The change in resonance frequency with porosity is illustrated in FIG. 4 which indicates that there is a minimum resonant frequency for a range of porosity levels. In the apparatus 30, however, it is only the deflection of the beam 21 that needs to be monitored. Conventional systems for detecting such deflection use a laser and a position sensitive detector to detect the deflection. The detection system is an external set-up and requires the laser to be optical aligned to the cantilever beam. The detection system used in the apparatus 30, on the other hand, uses the inherent optical detection system of the CD drive 29. The read/write head (RWH) 32 of the drive 29 is used to interrogate the cantilever sensor 20 and monitor the position of the reference surface 24. In addition the laser of the RWH may be used to control the temperature of the test and secondary chambers 5,6.

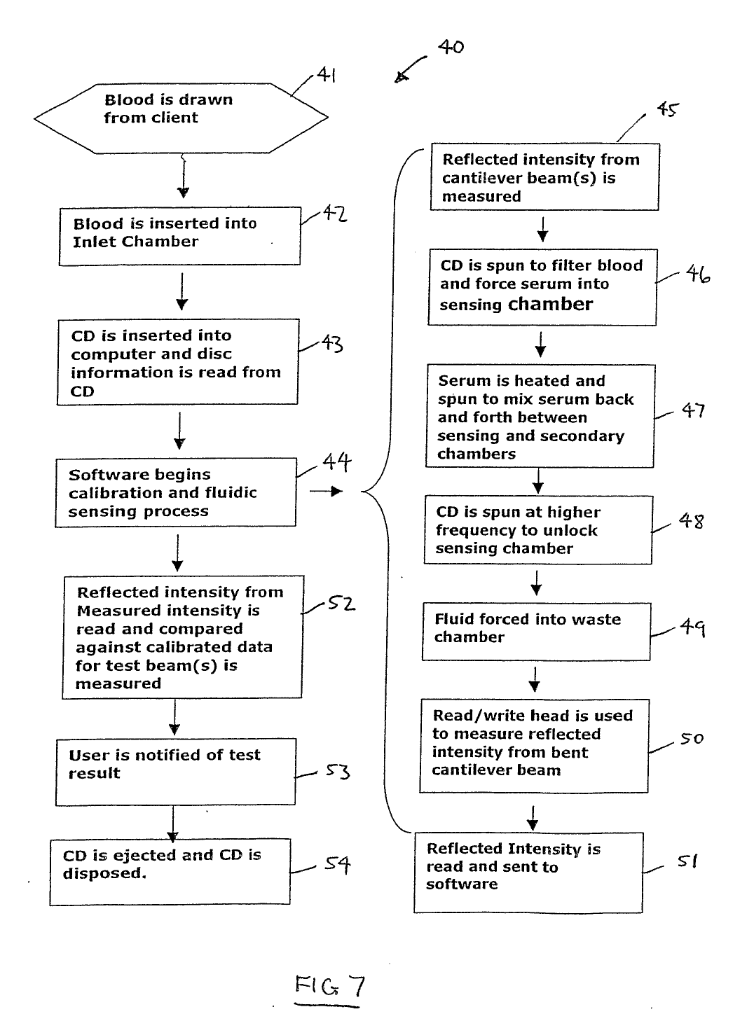

More particularly, to sense the deflection of the sensor 20, the RWH is moved over the position of the porous cantilever beam 21, as illustrated in FIG. 5. The CD device 1 can be rotating while sensing the deflection. The laser of the RWH is focused onto the cantilever beam 21 and the reflected intensity from the reference surface 24 of the beam 21 is measured prior to loading a test fluid into the test chamber 4, for calibration purposes. The test fluid is then caused to enter the test chamber 6 and subsequently exhausted to the waste chamber 7. The change in reflected intensity from the cantilever beam 21 after the test fluid has been removed from the test chamber 6 is measured. The change in reflected intensity is a measure of the sensor deflection. Secondary to this, the deflection can also be measured as a change in focus. When the laser is initially focused onto the beam 21 prior to loading the test fluid into the test chamber 6, the focus position can be measured. After the test fluid has been removed from the test chamber the beam 21 will have deflected and the reflective surface 24 will have moved out of focus. A graphical representation illustrating the affect of a change in focus on the measured intensity of reflected laser light is illustrated in FIG. 6. The change in focus is an indirect measure of the deflection and can be measured as a change in current or voltage output from the RWH.

### Application of Assay Device to Testing of Blood.

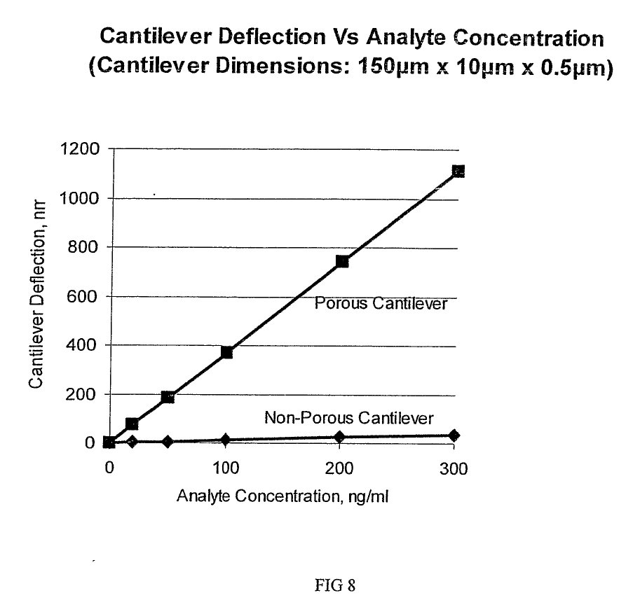

A detailed example of use of the assay device 1 and apparatus 20 is described with reference to FIG. 7. Specifically, a diagnostic test procedure 40 is shown as including a step 41 of drawing blood from a client and inserting the blood into the inlet port 4 of the device 1 at step 42. The CD device 1 is then inserted into a computer at step 43 and disc information is read from the CD. Relevant software is then employed at step 44 to initiate testing which commences at step 45 with the reflected intensity from the cantilever sensor 20 being measured for calibration purposes. The CD is then spun at step 46 to force the blood into the first channel 8 and through the filter 11, where cellular material is removed. The resulting serum is then passed through the secondary chamber 5 (if required) and into the test chamber 6. If required, the serum is then heated at step 47 by a laser of the RWH resulting in the serum being cycled back and forth between the test chamber 6 and secondary chamber 5 to improve interaction with the receptors. The CD is then spun at a higher angular velocity at step 48, to move the valve 13 into the open position so that the serum may exit the test chamber 6 and pass into the waste chamber 7 at step 49. The RWH may then be used to measure the reflected intensity of the displaced cantilever beam 21 at step 50 and the output of the RWH is then returned at step 51 for analysis at step 52, where the measured intensity is read and compared with calibrated data to determine the presence of a relevant chemical or molecule. The test results are then logged, a user notified of the results at step 53, and the CD ejected at step 54, as required. The CD may then be disposed of or stored for the purpose of a permanent record of the test result.

### Other Applications

The technology enables near patient health pathology to be performed, avoiding the need for use of expensive laboratory equipment and the associated delay in provision of results. Examples of the range of applications include:

Human Health Pathology
Detection of
   Prostate Specific Antigen
   Cardiac Enzymes
   Infectious diseases (Hepatitis, HIV)
   Snake bite venom

Environment Pathology
Detection of
   Leionella bacteria
   Hepatitis in water ways
   E-coli levels

Animal Health Pathology
Detection of
   Johne's disease

Fluid Quality Measurement
Detection of
   Wine fermentation

Industrial Measurement
Detection of electrical insulation deterioration.

The invention has been described by way of non-limiting example only and many modifications and variations may be made thereto without departing from the spirit and scope of the invention described.

The claims defining the invention are as follows:

1. An assay device having a rotatable platform with a test chamber and a sensor which undergoes displacement when subject to a particular substance such as a chemical, biological species or other organism.

2. An assay device as claimed in claim 1, wherein the sensor is a cantilever beam.

3. An assay device as claimed in claim 1 or 2, wherein the device includes microfluidic paths in communication with associated channels and the test chamber or an associated plurality of test chambers.

4. An assay device as claimed in claim 3, wherein the or each test chamber includes one or more cantilever beams.

5. An assay device as claimed in claim 1 or 2, wherein the sensor includes a porous section.

6. An assay device as claimed in claim 5, wherein the sensor is functionalised with receptors, antibodies, antigens or enzymes which will selectively attract and bond with the particular substance to be detected.

7. An assay device as claimed in claim 6, wherein the porous section is coated with a gold layer which attaches the receptors to the beam, functionalising the beam for bonding with preselected species or organism within the fluid in the test chamber.

8. An assay device as claimed in claim 1, wherein the sensor includes a surface to be monitored, which is subject to displacement upon movement of the sensor, the position of the monitored surface being monitored by an apparatus into which the assay device is loaded.

9. An assay device as claimed in claim 8, wherein the surface is a reflective surface.

10. An assay device as claimed in claim 8, wherein the apparatus is a CD drive connected to a computer allowing the position of the reflective surface to be determined and displayed by the computer.

11. An assay device as claimed in claim 1, wherein the assay device includes a micro-fluidic system for conveying a test fluid from an inlet port to the test chamber containing the cantilever beam and on to a waste chamber.

12. An assay device as claimed in claim 11, wherein the waste chamber is separated from the test chamber by a micro-mechanical valve which is actuated above a threshold angular velocity of the device.

13. An assay device as claimed in claim 11 or 12, wherein the device can accept the whole fluid to be tested and includes a filter for filtering material from the whole fluid after insertion into the inlet port to provide fluid in a form suitable for testing.

14. An assay device as claimed in claim 13, wherein the filter is formed of a porous silicon.

15. An assay device as claimed in claim 14, wherein the system includes provision for secondary chamber connected to the test chamber to allow the cycling of fluid between the secondary and test chamber.

16. An assay device as claimed in any previous claim, wherein the device is in the form of a compact disc (CD).

17. A test apparatus for receiving an assay device, as described above, including a drive unit for rotating the device and a read unit for monitoring the sensor.

18. A test apparatus as claimed in claim 17, wherein the apparatus is adapted to display information derived from the read unit.

19. A test apparatus as claimed in claim 17 or 18, wherein the apparatus is in the form of CD drive and the read unit forms part of an existing optical read/write head of the CD drive.

20. A test apparatus as claimed in any one of claims 17 to 19, wherein the apparatus is connected directly to a computer, on which is installed a computer program which controls the operation of the CD drive to initiate the filtering process, the transfer of fluid between chambers and the optical reading system to measure the displacement of the sensor.

21. A chemical assay method including introducing fluid to a sensing chamber on a rotatable platform, wherein the sensing chamber includes a sensor arranged for displacement upon detection of a particular substance, such as a selected molecule, within the chamber, and monitoring the sensor to detect the displacement.

22. A cantilever sensor as claimed in any one of the preceding claims.
